SEQUENCE LISTING (24 sequences)

SEQ ID NO: 1 - DNA, 20 bp, Artificial Sequence
  Oligonucleotide sequence (5′-3′)
gttggtggagcgatttgtct

SEQ ID NO: 2 - DNA, 20 bp, Artificial Sequence
  Oligonucleotide sequence (5′-3′)
cgctgagccagtcagtgtag

SEQ ID NO: 3 - DNA, 20 bp, Artificial Sequence
  Oligonucleotide sequence (5′-3′)
actggaacggtgaaggtgac

SEQ ID NO: 4 - DNA, 20 bp, Artificial Sequence
  oligonucleotide sequence (5′-3′)
gtggacttgggagaggactg

SEQ ID NO: 5 - DNA, 16 bp, Artificial Sequence
  oligonucleotide sequence (5′-3′)
gctgagtacgtcgtgg

SEQ ID NO: 6 - DNA, 17 bp, Artificial Sequence
  oligonucleotide sequence (5′-3′)
gtgctaagcagttggtg

SEQ ID NO: 7 - DNA, 16 bp, Artificial Sequence
  oligonucleotide sequence (5′-3′)
gcatctacggtgagcg

SEQ ID NO: 8 - DNA, 16 bp, Artificial Sequence
  oligonucleotide sequence (5′-3′)
aggggtttatcccggc

SEQ ID NO: 9 - DNA, 16 bp, Artificial Sequence
  oligonucleotide sequence (5′-3′)
aagagcatctacggtg

SEQ ID NO: 10 - DNA, 16 bp, Artificial Sequence
  oligonucleotide sequence (5′-3′)
gtttatcccggctgtc

SEQ ID NO: 11 - DNA, 20 bp, Artificial Sequence
  oligonucleotide sequence (5′-3′)
aagcgtggaaccttcatcga

SEQ ID NO: 12 - DNA, 23 bp, Artificial Sequence
  oligonucleotide sequence (5′-3′)
tcccggatcttctctttcttgtc

SEQ ID NO: 13 - DNA, 17 bp, Artificial Sequence
  oligonucleotide sequence (5′-3′)
tgggttttggggggacg

SEQ ID NO: 14 - DNA, 17 bp, Artificial Sequence
  oligonucleotide sequence (5′-3′)
tgtcagattcggggctc

SEQ ID NO: 15 - DNA, 20 bp, Artificial Sequence
  oligonucleotide sequence (5′-3′)
actccatcatttgtttcgtg

SEQ ID NO: 16 - DNA, 20 bp, Artificial Sequence
  oligonucleotide sequence (5′-3′)
tagtatccagatgcccacac

SEQ ID NO: 17 - DNA, 24 bp, Artificial Sequence
  oligonucleotide sequence (5′-3′)
acctgcagccttcactttcctcac

SEQ ID NO: 18 - DNA, 21 bp, Artificial Sequence
  oligonucleotide sequence (5′-3′)
ggcgatcacctccgtcacttg

SEQ ID NO: 19 - DNA, 20 bp, Artificial Sequence
  oligonucleotide sequence (5′-3′)
ccttctaacccatggctgtc

SEQ ID NO: 20 - DNA, 20 bp, Artificial Sequence
  oligonucleotide sequence (5′-3′)
tttttcaggtcctccactgc

SEQ ID NO: 21 - DNA, 20 bp, Artificial Sequence
  oligonucleotide sequence (5′-3′)
cgcatattcttcccagtggt

SEQ ID NO: 22 - DNA, 20 bp, Artificial Sequence
  oligonucleotide sequence (5′-3′)
gcgtcacttttgtcctggat

SEQ ID NO: 23 - DNA, 20 bp, Artificial Sequence
  oligonucleotide sequence (5′-3′)
aaattgccatcggtctgttc

SEQ ID NO: 24 - DNA, 20 bp, Artificial Sequence
  oligonucleotide sequence (5′-3′)
cccatagggctgataagcaa

(19) United States
(12) Patent Application Publication
(10) Pub. No.: US 2009/0269770 A1
(43) Pub. Date: Oct. 29, 2009

(54) METHODS FOR EVALUATION PROGNOSIS AND FOLLOW-UP OF DRUG TREATMENT OF PSYCHIATRIC DISEASES OR DISORDERS

(75) Inventors: Henry Silver, Haifa (IL); Moussa B.H. Youdim, Haifa (IL); Orly Weinreb, Haifa (IL)

Correspondence Address: BROWDY AND NEIMARK, P.L.L.C.;624 NINTH STREET, NW, SUITE 300, WASHINGTON, DC, 20001-5303, US

(73) Assignee: Technion Research and Development Foundation Ltd., Haifa (IL)

(21) Appl. No.: 12/429,527

(22) Filed: Apr. 24, 2009

Related U.S. Application Data
(60) Provisional application No. 61/047,629, filed on Apr. 24, 2008.

(51) Int. Cl. C12Q 1/68

(52) U.S. Cl. 435/6

(57) ABSTRACT

The present invention provides methods for evaluating the pharmacological efficacy of drugs or drug candidates in treatment of psychiatric diseases or disorders, particularly schizophrenia, and for predicting the efficacy of drugs or drug combinations indicated for treatment of both positive and negative symptoms of psychiatric diseases or disorders in an individual having such a disease or disorder. In both methods, the drugs or drug candidates evaluated are assessed for their ability to produce certain changes in the expression of specific genes in peripheral mononuclear cells in blood of psychiatric patients, which are similar to the changes obtained following treatments with reference drugs or drug combinations effective against both positive and negative symptoms of psychiatric diseases or disorders.

### TECHNICAL FIELD

The present invention relates to methods for evaluating the pharmacological efficacy of drugs or drug candidates in treatment of psychiatric diseases or disorders, particularly schizophrenia, and for predicting the efficacy of drugs or drug combinations indicated for treatment of both positive and negative symptoms of psychiatric diseases or disorders in an individual having such a disease or disorder.

### BACKGROUND ART

Schizophrenia is a serious mental illness characterized by impairments in the perception or expression of reality, most commonly manifesting as auditory hallucinations, paranoid or bizarre delusions or disorganized speech and thinking in the context of significant social or occupational dysfunction. Onset of symptoms typically occurs in young adulthood, with approximately 1% of the population worldwide affected. There is a well-known tendency for schizophrenia to run in families.

Dopamine antagonist antipsychotic drugs are the mainstay of schizophrenia treatment, but are not always effective, in particular against cognitive, motivational and emotional impairments, known as “negative symptoms”, of the disease. “Atypical” antipsychotics such as clozapine, olanzapine, risperidone and ziprazidone, are arguably more effective and better tolerated than the older drugs, but their effect is also limited (Lieberman et al., 2005; Murphy et al., 2006).

The simultaneous modification of multiple neurotransmitter systems may be advantageous in complex psychiatric disorders. This approach has lead to a search for multifunctional drugs (van Hes et al., 2003) and for drug combination as a strategy to improve efficacy. A successful example of this approach for the treatment of resistant symptoms of schizophrenia, depression and obsessive-compulsive disorder (OCD) is the coadministration of selective serotonin reuptake inhibitor (SSRI) antidepressants, e.g., fluvoxamine and fluoxetine, together with antipsychotics, which produce a synergistic therapeutic effect. A series of clinical studies have shown that this combination can improve negative symptoms of schizophrenia in patients unresponsive to antipsychotic alone (Silver and Nassar, 1992; Spina et al., 1994; Goff et al., 1995).

Improvement in negative symptoms can be detected within two weeks of starting treatment and is not explained by any changes in depressive symptoms or extrapyramidal side effects if present (Silver and Nassar, 1992; Silver et al., 1996, 2000, 2003; Silver and Shmugliakov, 1998). The augmenting effect is associated with the serotonergic system since maprotaline, an equally effective non-serotonergic antidepressant, did not improve negative symptoms (Silver and Shmugliakov, 1998). The mechanism of augmentation action is unknown and cannot be explained by the pharmacologic mechanisms of the individual drugs.

More effective treatments for schizophrenia and other psychiatric diseases are required but their development is limited by ignorance as to the biological causes and pathological processes. Discovery of biological substances, namely biomarkers, which can be related to treatment response, would advance development of new and more effective drugs.

### SUMMARY OF INVENTION

Preliminary clinical studies conducted in accordance with the present invention have shown specific and consistent changes in the expression level of certain genes, including genes encoding for G-protein-coupled receptors (GPCRs), in particular, cytokine receptors, regulators of G-protein signaling (RGS) and serotonergic receptors, in peripheral mononuclear cells (PMCs) from blood of schizophrenic patients following the addition of the antidepressant agent fluvoxamine, a selective serotonin reuptake inhibitor (SSRI), to ongoing antipsychotic treatment. These changes occurred following several days or weeks of the combined treatment in parallel to clinical improvement in negative symptoms (Chertkow et al., 2007), indicating that such changes may serve as biomarkers of treatment response, wherein certain patterns in the direction and timing of those changes may be used as a reference template to evaluate the pharmacological efficacy of drug candidates under clinical trials in treatment of psychiatric diseases or disorders, as well as to predict treatment response and progress of a patient having a psychiatric disease or disorder and treated with a drug or drug combination indicated for treatment of said psychiatric disease or disorder.

In one aspect, the present invention thus relates to a method for evaluating the pharmacological efficacy of a drug candidate in treatment of a psychiatric disease or disorder, said method comprising:
(i) administering to each individual in a group of patients having said psychiatric disease or disorder said drug candidate for a sufficient time period;
(ii) measuring expression levels of genes expressed in peripheral mononuclear cells (PMCs) in blood samples obtained from said patients at a first instant before the first administration of said drug candidate and at given second and third instants following the first administration of said drug candidate, thus obtaining a test gene expression profile expressing a representative relative level of each one of said genes at said second and third instants for said group of patients; and
(iii) comparing said test gene expression profile with either (a) a reference gene expression profile obtained as described in (ii) from a group of patients administered with a drug or drug combination effective against both positive and negative symptoms of psychiatric diseases or disorders, or (b) a predetermined reference gene expression profile expressing a representative relative level of each one of said genes at said second and third instants indicating an effective treatment against both positive and negative symptoms of psychiatric diseases or disorders,

wherein a significant similarity between said test gene expression profile and said reference gene expression profile or predetermined reference gene expression profile indicates that said drug candidate has a likelihood of being effective in treatment of said psychiatric disease or disorder.

In another aspect, the present invention relates to a method for predicting the efficacy of a drug or drug combination indicated for treatment of both positive and negative symptoms of psychiatric diseases or disorders in a patient having a psychiatric disease or disorder, said method comprising:
(i) administering to said patient said drug or drug combination for a sufficient time period;
(ii) measuring expression levels of genes expressed in peripheral mononuclear cells (PMCs) in blood samples obtained from said patient at a first instant before the first administration of said drug or drug combination and at given second and third instants following the first administration of said drug or drug combination, thus obtaining a test gene expression profile expressing a relative level of each one of said genes at said second and third instants for said patient; and
(iii) comparing said test gene expression profile with a predetermined reference gene expression profile expressing a representative relative level of each one of said genes at said second and third instants indicating an effective treatment against both positive and negative symptoms of psychiatric diseases or disorders,

wherein a significant similarity between said test gene expression profile and said predetermined reference gene expression profile indicates that said drug or drug combination has a likelihood of being effective in treatment of said patient.

In a further aspect, the present invention provides a kit for evaluating the pharmacological efficacy of a drug candidate in treatment of a psychiatric disease or disorder; or for predicting the efficacy of a drug or drug combination indicated for treatment of both positive and negative symptoms of psychiatric diseases or disorders in a patient having a psychiatric disease or disorder, said kit comprising:
(i) a list of genes expressed in peripheral mononuclear cells (PMCs);
(ii) a predetermined reference gene expression profile obtained from a group of patients administered with a drug or drug combination effective against both positive and negative symptoms of psychiatric diseases or disorders by measuring expression levels of said genes in blood samples obtained from said patients at a first instant before the first administration of said drug or drug combination and at given second and third instants following the first administration of said drug or drug combination, said profile expressing a representative relative level of each one of said genes at said second and third instants for said group of patients, indicating an effective treatment against both positive and negative symptoms of psychiatric diseases or disorders;
(iii) a set of oligonucleotides each comprising a nucleotide sequence complementary to a specific sequence of each one of said genes;
(iv) instructions for use; and optionally
(v) a container containing said drug or drug combination.

In preferred embodiments, the psychiatric disease or disorder is schizophrenia.

### BRIEF DESCRIPTION OF DRAWINGS

FIGS. 1A-1D show real-time RT-PCR analysis of CCR1 (1A), CCR7 (1B), IL8Ra (1C) and RGS7 (1D) mRNAs in PMCs from schizophrenic patients treated with an antipsychotic drug combined with the antidepressant agent fluvoxamine. Fluvoxamine (100 mg/day) was added in an open study format to the constant antipsychotic treatment of 6 patients suffering from chronic schizophrenia with persistent negative symptoms. Total RNA, isolated from PMCs of these patients at baseline (day 0, BL) as well as following 3 and 6 weeks (3W and 6W, respectively) of the combined treatment, was reverse transcribed. cDNA was amplified in real-time PCR using suitable primers for CCR1, CCR7, IL8Ra and RGS7, as described in the Experimental section hereinafter. The relative expression level of a given mRNA was assessed by normalizing to the reference gene peptidylprolyl isomerase B (cyclophilin B, PPIB). For each patient, the expression level of each one of the genes at baseline was arbitrarily set as 1, and the gene expression levels at 3 and 6 weeks were calculated relative to baseline. Lines connecting points indicate samples of the same patient. Horizontal lines indicate group means. Student's t-test *p<0.05; **p<0.01 for 3 or 6 weeks of fluvoxamine add-on compared with baseline.

FIGS. 2A-2B show real-time RT-PCR analysis of GABA_{A}β2 (2A) and PKCβ2 (2B) mRNAs in PMCs from schizophrenic patients treated with an antipsychotic drug combined with the antidepressant agent fluvoxamine. Fluvoxamine (100 mg/day) was added in an open study format to the constant antipsychotic treatment of 8 patients suffering from chronic schizophrenia with persistent negative symptoms. Total RNA, isolated from PMCs of these patients at baseline (day 0, BL) as well as following 1, 3 and 6 weeks (1W, 3W and 6W, respectively) of the combined treatment, was reverse transcribed. cDNA was amplified in real-time PCR using suitable primers for GABA_{A}β2 and PKCβ2, as described in the Experimental section. The relative expression level of a given mRNA was assessed by normalizing to the reference gene PPIB. For each patient, the expression level of each one of the genes at baseline was arbitrarily set as 1, and the gene expression levels at 3 and 6 weeks were calculated relative to baseline. Lines connecting points indicate samples of the same patient. Dash line indicates average of the samples of different objects. Student's t-test *p<0.05; **p<0.01 for 1, 3 or 6 weeks of fluvoxamine add-on compared with baseline.

### MODES FOR CARRYING OUT THE INVENTION

As stated above, the present invention relates to both (1) a method for evaluating the pharmacological efficacy of a drug candidate in treatment of a psychiatric disease or disorder, as well as (2) a method for predicting the efficacy of a drug or drug combination indicated for treatment of both positive and negative symptoms of psychiatric diseases or disorders in a patient having a psychiatric disease or disorder. It should be noted that the various definitions, terms and phrases used herein refer to both of these methods.

In one aspect, the present invention relates to a method for evaluating the pharmacological efficacy of a drug candidate in treatment of a psychiatric disease or disorder, as defined above. This method may be utilized in clinical trials in which the pharmacological efficacy of a drug candidate in treatment of a psychiatric disease or disorder is evaluated using a group of patients having said psychiatric disease or disorder, wherein each one of the patients participating in the clinical trial serves as his own control. Such clinical trials may be carried out wherein a first group of patients is administered with the drug candidate and a second group of patients is administered with a reference drug or drug combination effective against both positive and negative symptoms of psychiatric diseases or disorders or, alternatively, with a placebo. As a consequence, the reference gene expression profile indicating an effective treatment against both positive and negative symptoms of psychiatric diseases or disorders may be established as part of this method or, alternatively, may be predetermined.

The term “drug candidate”, as used herein, refers to any molecule being evaluated for treatment of a psychiatric disease or disorder, which may be either a drug approved for treatment of human against an indication other than a psychiatric disease or disorder, or a chemical molecule currently being developed as a drug for treatment of a psychiatric disease or disorder.

The phrase “drug or drug combination effective against both positive and negative symptoms of psychiatric diseases or disorders” or “reference drug or drug combination”, used herein interchangeably, refers to any drug or drug combination that is effective against both positive symptoms, i.e., hallucinations, delusions and racing thoughts, which generally respond to antipsychotic medicines, as well as negative symptoms, i.e., apathy, lack of emotion and poor or nonexistant social functioning, associated with psychiatric diseases or disorders. In view of these properties, such drug or drug combination can thus principally be used in treating patients with treatment-resistant schizophrenia, a term generally used for the failure of symptoms to satisfactorily respond to at least two different antipsychotics.

In one embodiment, the drug combination effective against both positive and negative symptoms of psychiatric diseases or disorders is a combination of an antipsychotic agent and an antidepressant agent functioning pharmacologically as a selective serotonin reuptake inhibitor (SSRI).

Non-limiting examples of antipsychotic agents include the atypical antipsychotic drugs risperidone (Risperdal®), olanzapine (Zyprexa®), ziprasidone (Geodone®) and clozapine; the typical antipsychotic drugs haloperidol, perphenazine and trifluperazine (Eskazinyl®); the antipsychotic drug amisulpride (Solian®); and a thioxanthene derivative such as the typical antipsychotic drugs chlorprothixene and thiothixene (Navane®), and the typical antipsychotic neuroleptic drugs flupentixol (Depixol® or Fluanxol®) and zuclopenthixol (Cisordinol®, Clopixol® or Acuphase®), available as zuclopenthixol decanoate, zuclopenthixol acetate and zuclopenthixol dihydrochloride.

Examples of antidepressant agents, without limitation, include fluoxetine, an antidepressant of the SSRI class (Prozac®); or fluvoxamine, an antidepressant which functions pharmacologically as an SSRI (Luvox®).

In a preferred embodiment, the drug combination effective against both positive and negative symptoms of psychiatric diseases or disorders is a combination of the typical antipsychotic drug haloperidol and the antidepressant agent fluvoxamine.

The administration to each one of the patients according to this method, either of the drug candidate or, alternatively, of the reference drug or drug combination, is performed in accordance with the specific clinical trial protocol. In particular, the administration of both the drug candidate and the reference drug or drug combination may be performed by any suitable route such as, without being limited to, intravenously, intramuscularly, orally, parenterally, rectally or transdermally, wherein the dosage and administration intervals, i.e., daily, weekly, monthly etc., are determined according to the clinical trial protocol.

The phrase “genes expressed in peripheral mononuclear cells”, as used herein, refers to any gene which transcript can be found in RNA extracted from these cells using conventional methods, e.g., as described in the Experimental section hereinafter.

In one embodiment, the genes expressed in peripheral mononuclear cells (PMCs) according to the present invention encode for G-protein-coupled receptors (GPCRs), proteins involved in primary metabolism, calcium signaling regulators or cell signaling regulators.

Examples of G-protein-coupled receptors (GPCRs) and associated signaling regulators, without being limited to, include chemokine receptors, chemokine-like receptors, regulators of G-protein signaling, serotonin (5-hydroxytryptamine, 5-HT) receptors, guanine nucleotide-binding protein G(i) subunit alpha-2, also known as adenylate cyclase-inhibiting G alpha protein, guanine nucleotide-binding protein G(q) subunit alpha, also known as guanine nucleotide-binding protein q-polypeptide or GNAQ, receptor of activated protein kinase C 1 (RACK1) and gamma aminobutyric acid (GABA)_{A}β2.

Examples of chemokine receptors, without limitation, include chemokine (C-C motif) receptor 1-10, i.e., CCR1, CCR2, CCR3, CCR4, CCR5, CCR6, CCR7, CCR8, CCR9 and CCR10, chemokine (C-C motif) receptor-like 1 (CCRL1) and interleukin 8 receptor alpha (IL8Rα).

A non-limiting example of chemokine-like receptors is chemokine-like receptor 1 (CMKLR1).

Non-limiting examples of regulators of G-protein signaling include regulator of G-protein signaling 2, 4 and 7, i.e., RGS2, RGS4 and RGS7, respectively.

Examples of serotonin receptors, without limitation, include 5-HT_{2A}, 5HT_{3A}, 5HT_{3B} and 5HT₇.

Examples of proteins involved in primary metabolism, without being limited to, include nuclear receptor-related 1 (NURR1), phosphatidylinositol transfer protein alpha isoform (PI-TP-alpha), acid beta-galactosidase (GLB-1) and ubiquitin.

Examples of calcium signaling regulators, without limitation, include 1,4,5-trisphosphate 3-kinase and neurogranin (NRGN).

Examples of cell signaling regulators, without being limited to, include protein kinase C (PKC)β2, extracellular signal-regulated kinase 1 (ERK1) and extracellular signal-regulated kinase 2 (ERK2).

According to this method, the expression level of each one of the genes is measured in PMCs in blood samples obtained from the patients administered either with the drug candidate which pharmacological efficacy in treatment of a psychiatric disease or disorder is evaluated, or with the reference drug or drug combination as defined above.

The expression levels of the various genes measured according to this method are determined at three given instants of time, wherein the first instant is before the first administration of the drug candidate being evaluated; and the second and the third instants are at certain points in time after the first administration. As exemplified herein, the changes observed in the expression level of each one of the genes measured occurred several days or weeks after the first administration of the antipsychotic-SSRI drug combination, in parallel to clinical improvement in negative symptoms. Thus, in most cases, both the second and the third instants are up to 8 weeks following the first administration of the drug candidate; however, in some cases, a longer duration of administration may be required, hence, the second and/or the third instant may be at a certain point in time that is more than 8 weeks following the first administration.

In cases wherein a second group of patients is being administered with a reference drug or drug combination, the expression levels of the various genes measured according to this method with respect to this group are determined at instants of time as defined for the first group of patients administered with the candidate drug, i.e., wherein the first instant is before the first administration of the reference drug or drug combination; and the second and the third instants are at points in time after the first administration as defined for the first group.

In one embodiment, the second and third instants are up to 8 weeks following the first administration of said drug candidate. In preferred embodiments, the second and third instants are 2 to 4 and 5 to 7 weeks, respectively, following the first administration of said drug candidate, more preferably about 3 and about 6 weeks, respectively, following the first administration of said drug candidate.

It should be noted that the expression levels of each one of the various genes measured according to this method at any one of the instants may be carried out using any suitable technique known in the art, e.g., as described in the Experimental section hereinafter.

In all cases, and although each one of the patients treated serves as his own control, gene expression levels are measured and compared with the level of a control gene which is not influenced neither by the drug candidate being evaluated nor by the reference drug or drug combination. Non-limiting examples of control genes include glyceraldehyde-3-phosphate dehydrogenase (GAPDH), β-actin, peptidylpropyl isomerase B (cyclophilin B, PPIB), phosphomannomutase (PPMM) and 18S ribosomal RNA. In a preferred embodiment, the control gene is PPIB.

The term “gene expression profile”, as used herein, refers to a profile showing the relative expression level of each one of the genes expressed in PMCs and measured in blood samples obtained from a patient administered according to the method of the present invention either with the drug candidate being evaluated, or with the reference drug or drug combination, at a second and a third instant as defined above compared with its level at the first instant, i.e., before the first administration of said drug candidate or the reference drug or drug combination, and at the third instant compared with its level at the second instant. As defined herein, a gene expression profile includes at least three genes expressed in PMCs as defined above, preferably at least five such genes, more preferably at least eight such genes.

The relative expression level of each one of the genes measured at the second and the third instants is represented by “increase”, indicating that the expression level of said gene at the specific instant is increased compared with its expression level at the first instant by at least 30%, preferably at least 40%, more preferably about 50%; “decrease”, indicating that the expression level of said gene at the specific instant is decreased compared with its expression level at the first instant by at least 30%, preferably at least 40%, more preferably about 50%; or “no change”, indicating that the expression level of said gene at the specific instant is neither increased or decreased as defined above.

The relative expression level of each one of the genes measured at the third instant compared with its level at the second instant is determined based on the relative expression levels of said gene at these two instants as defined hereinabove. In particular, the relative expression level of a gene measured at the third instant compared with its level at the second instant is represented by “increase”, in cases wherein the relative expression level of said gene is represented by “no change” at the second instant and by “increase” at the third instant, or the relative expression level of said gene is represented by “decrease” at the second instant and by either “increase” or “no change” at the third instant; “decrease”, in cases wherein the relative expression level of said gene is represented by “no change” at the second instant and by “decrease” at the third instant, or the relative expression level of said gene is represented by “increase” at the second instant and by either “decrease” or “no change” at the third instant; or “no change”, in cases wherein the relative expression levels of said gene at the second and the third instant are identical.

The phrase “gene expression profile expressing a representative relative level of each one of said genes at said second and third instants” or “representative relative gene expression profile”, as used herein interchangeably, refers to a gene expression profile established for a group of patients administered either with the drug candidate being evaluated or with the reference drug or drug combination, based on the gene expression profile of each one of the patients in this group, showing the representative relative expression levels of each one of the genes measured according to the method of the present invention in blood samples obtained from each one of the patients in this group, at a second and a third instant as defined above.

The representative relative gene expression profile defined hereinabove may be established using any suitable algorithm.

In one embodiment and as exemplified herein, the representative relative expression levels of each one of the genes measured at the second and the third instants are represented by “increase”, indicating that the expression level of said gene at the specific instant in most of the patients in the group is increased compared with its expression level at the first instant; “decrease”, indicating that the expression level of said gene at the specific instant in most of the patients in the group is decreased compared with its expression level at the first instant; or “no change”, indicating that the expression level of said gene at the specific instant in most of the patients in the group is “no change”. As defined herein, the term “most of the patients” refers to at least 50%, preferably at least 60%, more preferably at least 65%, most preferably at least 75%, of the patients in the group administered as defined above.

The term “test gene expression profile” refers to a representative relative gene expression profile as defined hereinabove, established for a group of patients administered with the drug candidate being evaluated. Similarly, the term “reference gene expression profile” refers to a representative relative gene expression profile, established for a group of patients administered with the reference drug or drug combination. As described above, clinical trials utilizing this method may be carried out wherein the reference gene expression profile indicating an effective treatment against both positive and negative symptoms of psychiatric diseases or disorders is established as part of this method or, alternatively, is predetermined. Thus, the term “predetermined reference gene expression profile” refers to a predetermined representative relative gene expression profile indicating an effective treatment against both positive and negative symptoms of psychiatric diseases or disorders.

The phrase “significant similarity between the profiles” refers to a situation in which the pattern of changes observed in the test gene expression profile at the second and the third instants with respect to at least 3 of the genes included in the profiles is identical to the pattern of changes observed with respect to these genes in the reference gene expression profile, either established as part of this method or predetermined. In fact, the likelihood of the drug candidate evaluated being effective is considered to increase with the increase in the number of genes which are altered in the direction and timing defined by the reference gene expression profile, wherein a total similarity between the profiles indicates a very high likelihood of the drug candidate evaluated being effective.

In a preferred embodiment, the genes expressed in PMCs encode for certain G-protein-coupled receptors and cell signaling regulators, in particular, for the GPCRs CC chemokine receptor 1 (CCR1), CC chemokine receptor 5 (CCR5), CC chemokine receptor 7 (CCR7), CC chemokine receptor-like 1 (CCRL1) interleukin 8 receptor alpha (IL8Rα); chemokine-like receptor 1 (CMKLR1); regulator of G-protein signaling 7 (RGS7); serotonin receptor 5-HT_{2A}, serotonin receptor 5-HT₇ and GABA_{A}β2; and for the cell signaling regulator PKCβ2.

In a most preferred embodiment, the genes expressed in PMCs encode for CCR1, CCR5, CCR7, CCRL1, IL8Rα, CMKLR1, RGS7, 5-HT_{2A}, 5-HT₇, GABA_{A}β2 and PKCβ2; the second and third instants are about 3 and about 6 weeks, respectively, following the first administration of the drug candidate; and the reference gene expression profile to which the test gene expression profile is compared, either established as part of this method or predetermined, shows a decrease in the CCR1, CCRL1, CMKLR1, IL8Rα, RGS7, 5-HT_{2A}, 5-HT₇ and PKCβ2 gene expression levels at the second or third instant relative to the first instant; an increase in the CCR5 and GABA_{A}β2 gene expression levels at the second or third instant relative to the first instant; and an increase in CCR7 and CCRL1 gene expression levels at the third instant relative to the second instant.

The psychiatric disease or disorder according to the present invention may be any psychiatric or neuropsychiatric disease or disorder which includes disturbances in motivational, emotional or cognitive function, i.e., “negative symptoms”, as part of the clinical syndrome, such as schizophrenia, obsessive-compulsive disorder (OCD), major depression, bipolar disorder or dementia accompanied, i.e., complicated, by aggression or affective disorder, i.e., mental disorder characterized by dramatic changes or extremes of mood, such as manic (elevated, expansive or irritable mood with hyperactivity, pressured speech and inflated self-esteem), depressive (dejected mood with disinterest in life, apathy, sleep disturbance, agitation and feelings of worthlessness or guilt) episodes, or combinations thereof. In a preferred embodiment, the psychiatric disease or disorder is schizophrenia.

The various studies described in detail in the Example section hereinafter show consistent gene expression changes in PMCs of schizophrenic patients undergoing combined antipsychotic-fluvoxamine treatment, indicating that PMCs may be useful in investigating the mechanism of action of these drugs in clinical settings consistent with other reports (Kronfol and Remick, 2000; Avissar et al., 2001; Ilani et al., 2001; Rothermundt et al., 2001; Tardito et al., 2001; Gladkevich et al., 2004; Tang et al., 2005; Bowden et al., 2006; Liew et al., 2006). Moreover, the within-subject design of the procedure established in the studies conducted, i.e., the fact that each one of the individuals treated served as his own specific control, reduced the potential confounds due to the heterogeneity of schizophrenic disease and highlighted the treatment-related changes.

While the validity of peripheral changes in genes expression as indicators of brain processes is still debated, there is evidence of crosstalk between neurotransmitters and immune-related proteins in brain and blood (Grimaldi and Fillion, 2000; Kronfol and Remick, 2000; Rothermundt et al., 2001; Wilson et al., 2002; Gladkevich et al., 2004). In addition, RGS family members (Larminie et al., 2004), most cytokines (Kronfol and Remick, 2000) and serotonin receptors (Grimaldi and Fillion, 2000) can be synthesized and function within the central nervous system, as well as in lymphocytes.

This fulfills a fundamental condition for correlation between brain and periphery, i.e., the criterion of expression of gene in both compartments (Sullivan et al., 2006). Some of these genes, in particular, 5-HT_{2A} receptor (Dean et al., 1999), IL-1 receptor antagonist (Toyooka et al., 2003), RGS7 (Mirnics et al., 2001; Bowden et al., 2007) and the neural specific protein neurogranin (Broadbelt et al., 2006), have been reported to be abnormally expressed in the brains of schizophrenic patients. Taken together, it is plausible to consider that the peripheral gene changes observed in the studies described in the Example section following combined antipsychotic-fluvoxamine treatment may reflect, at least in part, relevant brain processes.

The broad preliminary microarray screening showed consistent changes in several gene groups known to be affected by antidepressant and antipsychotic action, including G-proteins (GNAI2, GNAQ) (Avissar et al., 2001), protein kinase C (RACK1), phosphotidyl inositol pathway (PI-TP-α, IP3K) (Opeskin et al., 1996) and neurogranin (Broadbelt et al., 2006). Subsequently, we investigated the changes in GPCR-related transcripts, of which the most significant changes after the addition of fluvoxamine were in cytokine receptors, RGS protein and serotonergic receptors that are of interest in light of evidence linking them to schizophrenia. Chemokines and the broader family of cytokines have been associated with various brain activities (Kronfol and Remick, 2000; Rothermundt et al., 2001) and implicated in the pathology of schizophrenia and its treatment (Barak et al., 1995; Muller et al., 1999; Kim et al., 2000; Kronfol and Remick, 2000; Zhang et al., 2002). IL-8, essential for the directional migration of leukocytes, is increased in the serum of unmedicated chronic schizophrenic patients (Erbagci et al., 2001; Maes et al., 2002; Zhang et al., 2002; Brown et al., 2004; Zhang et al., 2004). The current finding that IL-8 receptor transcript level is reduced after adding fluvoxamine raises the possibility that the mechanism of action of the combined treatment opposes the pathological increase in the ligand concentration.

Reduction in RGS7 gene expression following the addition of fluvoxamine to ongoing antipsychotic treatment was of interest since RGS proteins modulate neurotransmitter-GPCR interactions and may be abnormal in the brains of schizophrenic patients (Mirnics et al., 2001; Bowden et al., 2007). RGS7, a short-lived GTPase-activating protein (Kim et al., 1999), is enriched in the human striatum and cerebellum (Larminie et al., 2004), areas of relevance to schizophrenia. It has been implicated in CNS dysfunctions (Benzing et al., 1999; Gold et al., 2002) and may reduce 5-HT_{2A} receptor mediated signaling (Ghavami et al., 2004). The finding that 5-HT_{2A} expression was decreased after combined antipsychotic-fluvoxamine treatment is consistent with evidence of reduced 5-HT_{2A} expression in rats administered the atypical antipsychotic olanzapine (Huang et al., 2006), and raises the possibility that the mechanism may involve changes in RGS7 modulation of the 5-HT_{2A} receptor.

In view of the aforesaid, the present invention particularly relates to a method for evaluating the pharmacological efficacy of a drug candidate in treatment of schizophrenia, said method comprising:
(i) administering to each individual in a group of patients having schizoprenia said drug candidate for a sufficient time period;
(ii) measuring expression levels of the genes CCR1, CCR5, CCR7, CCRL1, CMKLR1, IL8Rα, RGS7, 5-HT_{2A}, 5-HT₇, GABA_{A}β2 and PKCβ2, in peripheral mononuclear cells (PMCs) in blood samples obtained from said patients at a first instant before the first administration of said drug candidate and at second and third instants about 3 and 6 weeks, respectively, following the first administration of said drug candidate, thus obtaining a test gene expression profile expressing a representative relative level of each one of said genes at said second and third instants for said group of patients; and
(iii) analyzing said test gene expression profile,

wherein a decrease in the CCR1, CCRL1, CMKLR1, IL8Rα, RGS7, 5-HT_{2A}, 5-HT₇ and PKCβ2 gene expression levels at said second or third instant relative to said first instant; together with an increase in the CCR5 and GABA_{A}β2 gene expression levels at said second or third instant relative to said first instant; and together with an increase in CCR7 and CCRL1 gene expression levels at said third instant relative to said second instant, indicate that said drug candidate has a likelihood of being effective in treatment of schizophrenia.

The present invention provides for the first time valid biological markers of treatment response in psychiatric diseases or disorders such as schizophrenia. In particular, the invention identifies a number of biomarkers expressed on PMCs, and establishes a reference pattern of changes in response to effective treatment against both positive and negative symptoms of psychiatric diseases or disorders, to which drug candidates are compared.

In other words, the present invention uses proven clinical effectiveness against both positive symptoms as well as negative symptoms of schizophrenia, resistant to currently available standard treatments, as the ultimate criterion for evaluating the pharmacological efficacy of drug candidates in treatment of schizophrenia and other psychiatric diseases or disorders. The concept of the invention is based on the principle that specific changes in the expression level of certain genes expressed in PMCs, which are not associated with antipsychotic treatment directed specifically against positive symptoms of the psychiatric disease or disorder, but are consistently associated with clinically effective combined SSRI-antipsychotic treatments, are used as a reference profile when evaluating the pharmacological efficacy of drug candidates.

Monitoring and analyzing the changes in the proposed profile of biomarkers may further be used to predict the onset of clinical improvement of a specific treatment with a drug or drug combination indicated for treatment of both positive and negative symptoms of psychiatric diseases or disorders, and to follow the progress of said treatment in an individual having a psychiatric disease or disorder as defined hereinabove and treated with said drug or drug combination.

Currently, there are no reliable objective biological measures, which can predict the response of a patient having a psychiatric disease or disorder to a given drug or provide objective measures of the effectiveness of said drug. Current clinical assessments rely on observation of behavioral change that is difficult to objectively assess and measure, and on self-report of patients themselves. Furthermore, changes in behavior and symptoms of the patients require large changes in complex systems and occur much later than the biochemical changes being associated with the processes, which ultimately produce these improvements in behavior and symptoms. The presence of objective biological markers can thus allow early prediction, e.g., within days up to several weeks, of treatment effectiveness and provide objective measures to follow treatment progress.

Thus, in another aspect, the present invention relates to a method for predicting the efficacy of a drug or drug combination indicated for treatment of both positive and negative symptoms of psychiatric diseases or disorders in a patient having a psychiatric disease or disorder, said method comprising:
(i) administering to said patient said drug or drug combination for a sufficient time period;
(ii) measuring expression levels of genes expressed in peripheral mononuclear cells (PMCs) in blood samples obtained from said patient at a first instant before the first administration of said drug or drug combination and at given second and third instants following the first administration of said drug or drug combination, thus obtaining a test gene expression profile expressing a relative level of each one of said genes at said second and third instants for said patient; and
(iii) comparing said test gene expression profile with a predetermined reference gene expression profile expressing a representative relative level of each one of said genes at said second and third instants indicating an effective treatment against both positive and negative symptoms of psychiatric diseases or disorders,

wherein a significant similarity between said test gene expression profile and said predetermined reference gene expression profile indicates that said drug or drug combination has a likelihood of being effective in treatment of said patient.

In one embodiment, the second and third instants are up to 8 weeks following the first administration of said drug or drug combination, namely during the first 8 weeks of the treatment period. In preferred embodiments, the second and third instants are 2 to 4 and 5 to 7 weeks, respectively, following the first administration of said drug or drug combination, more preferably about 3 and about 6 weeks, respectively, following the first administration of said drug or drug combination.

In a preferred embodiment, the genes expressed in PMCs encode for certain G-protein-coupled receptors and cell signaling regulators, in particular, for the GPCRs CCR1, CCR5, CCR7, CCRL1, IL8Rα, CMKLR1, RGS7, 5-HT_{2A}, 5-HT₇ and GABA_{A}β2; and for the cell signaling regulator PKCβ2.

In a most preferred embodiment, the genes expressed in PMCs encode for CCR1, CCR5, CCR7, CCRL1, IL8Rα, CMKLR1, RGS7, 5-HT_{2A}, 5-HT₇, GABA_{A}β2 and PKCβ2; the second and third instants are about 3 and about 6 weeks, respectively, following the first administration of the drug candidate; and the predetermined reference gene expression profile to which the test gene expression profile is compared, shows a decrease in the CCR1, CCRL1, CMKLR1, IL8Rα, RGS7, 5-HT_{2A}, 5-HT₇ and PKCβ2 gene expression levels at the second or third instant relative to the first instant; an increase in the CCR5 and GABA_{A}β2 gene expression levels at the second or third instant relative to the first instant; and an increase in CCR7 and CCRL1 gene expression levels at the third instant relative to the second instant.

Thus, the present invention particularly relates to a method for predicting the efficacy of a drug or drug combination indicated for treatment of both positive and negative symptoms of psychiatric diseases or disorders in a patient having schizophrenia, said method comprising:
(i) administering to said patient said drug or drug combination for a sufficient time period;
(ii) measuring expression levels of the genes CCR1, CCR5, CCR7, CCRL1, CMKLR1, IL8Rα, RGS7, 5-HT_{2A}, 5-HT₇, GABA_{A}β2 and PKCβ2, in peripheral mononuclear cells (PMCs) in blood samples obtained from said patient at a first instant before the first administration of said drug or drug combination and at second and third instants about 3 and 6 weeks, respectively, following the first administration of said drug or drug combination, thus obtaining a test gene expression profile expressing a relative level of each one of said genes at said second and third instants for said patient; and
(iii) analyzing said test gene expression profile,

wherein a decrease in the CCR1, CCRL1, CMKLR1, IL8Rα, RGS7, 5-HT_{2A}, 5-HT₇ and PKCβ2 gene expression levels at said second or third instant relative to said first instant; together with an increase in the CCR5 and GABA_{A}β2 gene expression levels at said second or third instant relative to said first instant; and together with an increase in CCR7 and CCRL1 gene expression levels at said third instant relative to said second instant, indicate that said drug or drug combination has a likelihood of being effective in treatment of said patient.

In a further aspect, the present invention provides a kit for evaluating the pharmacological efficacy of a drug candidate in treatment of a psychiatric disease or disorder; or for predicting the efficacy of a drug or drug combination indicated for treatment of both positive and negative symptoms of psychiatric diseases or disorders in a patient having a psychiatric disease or disorder, said kit comprising:
(i) a list of genes expressed in peripheral mononuclear cells (PMCs);
(ii) a predetermined reference gene expression profile obtained from a group of patients administered with a drug or drug combination effective against both positive and negative symptoms of psychiatric diseases or disorders by measuring expression levels of said genes in blood samples obtained from said patients at a first instant before the first administration of said drug or drug combination and at given second and third instants following the first administration of said drug or drug combination, said profile expressing a representative relative level of each one of said genes at said second and third instants for said group of patients, indicating an effective treatment against both positive and negative symptoms of psychiatric diseases or disorders;
(iii) a set of oligonucleotides each comprising a nucleotide sequence complementary to a specific sequence of each one of said genes;
(iv) instructions for use; and optionally
(v) a container containing said drug or drug combination.

The kit of the present invention can be used for carrying out both of the methods defined above, i.e., (1) the method for evaluating the pharmacological efficacy of a drug candidate in treatment of a psychiatric disease or disorder; and (2) the method for predicting the efficacy of a drug or drug combination indicated for treatment of both positive and negative symptoms of psychiatric diseases or disorders in a patient having a psychiatric disease or disorder.

As described in detail hereinabove, in both of these methods, the expression levels of genes expressed in PMCs are measured in blood samples of patients treated with either the drug candidate according to the method of (1), or the drug or drug combination indicated for treatment of both positive and negative symptoms of psychiatric diseases or disorders according to the method of (2), at three different instants before and during the treatment, and a test gene expression profile for either a group of patients according to the method of (1) or a sole patient according to the method of (2) is obtained.

As further described, whereas the method of (2) is directed at predicting the efficacy of a certain medical treatment in a sole patient having a psychiatric disease or disorder, the method of (1) is used in clinical trials directed at evaluating the pharmacological efficacy of a drug candidate in treatment of a psychiatric disease or disorder, using a group of patients having such a disease or disorder. In other words, whereas the test gene expression profile obtained according to the method of (2) is compared with a predetermined reference gene expression profile, the test gene expression profile obtained according to the method of (1) is compared with either a reference gene expression profile established as a part of that method or, alternatively, a predetermined reference gene expression profile.

Thus, the kit of the present invention comprises both a list of genes expressed in PMCs, the expression levels of which are measured, as well as a predetermined reference gene expression profile to which the established test gene expression profile is compared according to the method of (2), or may be compared according to the method of (1).

In cases this kit is used for carrying out the method of (1), in particular in clinical trials wherein a first group of patients is treated with a drug candidate and a second group of patients is treated with a reference drug or drug combination, said drug or drug combination is further provided as a part of this kit. The drug or drug combination optionally comprised in this kit may be provided in any suitable form, e.g., as tablets, pills, powder, soft gelatin capsules, lozenges, syrup and emulsion, and may be packed in any suitable container such as, without being limited to, a packaging box, ampoule of vial.

In cases wherein the reference drug or drug combination is provided with the kit, in order to assure the quality of the assay performed, the reference gene expression profile obtained is first compared with the predetermined reference gene expression profile provided. Providing that the reference gene expression profile obtained is identical to, i.e., of total similarity with, the predetermined reference gene expression profile provided, the comparison between the test gene expression profile and either the reference gene expression profile or the predetermined reference gene expression profile can then be performed as described above.

In order to produce the test gene expression profile and optionally the reference gene expression profile, the patients participating in the clinical trial according to the method of (1), or the sole patient examined according to the method of (2), are treated as defined in step (i) of these methods, and expression levels of the genes indicated are measured in PMCs in blood samples obtained from said patient/s as defined in step (ii) of said methods.

The isolation of PMCs from blood samples obtained from the patient/s treated according to the methods of this invention, as well as the extraction of total RNA from said PMCs, may be carried out using any suitable technology known in the art, e.g., as described in the Experimental section hereinafter. Examples of materials and tools that may be useful for these purposes include anticoagulants such as ethylenediaminetetraacetic acid (EDTA) and EDTA-coated tubes, materials that may be used for blood separation such as Ficoll (Sigma); and RNA extraction reagents such as TriReagent (Sigma). The analysis of the expression levels of each one of the genes of interest, according to the methods of this invention, may be carried out by any suitable technology known in the art such as, without being limited to, real-time quantitative reverse transcribed PCR, as exemplified in the Experimental section hereinafter.

The oligonucleotides provided as a part of the kit of the present invention are, in fact, primers that can be used for the detection of said genes expressed in PMCs, wherein each one of said primers is complementary to a specific sequence in one of said genes. The primers provided may be any suitable primers enabling the detection of the specific genes the expression levels of which are measured. Non-limiting examples of primers complementary to specific sequences of the genes 18S, β-actin, GAPDH, PPIB, PPMM, IL8R, CCR7, CCR1, RGS7, GABA_{A}β2 and PKCβ2, are those of SEQ ID NOs. 1-24, listed in the Experimental section hereinafter.

The invention will now be illustrated by the following non-limiting Examples.

### EXAMPLES

### Experimental

### 1. Experimental Protocol

Patients suffering from chronic schizophrenia (DSM IV criteria) with at least 2 years of illness (range 2-17 years) and persistent negative symptoms were chosen from an Israeli mental health center, as shown in Table 1. To qualify for inclusion in the study, these patients were required to be on constant antipsychotic medication for at least 6 months prior to the study and on an unchanged dose for at least 4 weeks before entry.

TABLE 1
Patients data

| Patient | Sex/age | Diagnosis | Antipsychotic | Dose (mg) |
|---|---|---|---|---|
| 1 | M/35 | Paranoid schizophrenia | Risperidone^{a} | 6 |
| 2 | M/27 | Paranoid schizophrenia | Olanzapine^{a} | 10 |
| 3 | M/43 | Paranoid schizophrenia | Zuclopenthixol | 100 |
| | | | dihydroxate^{b} | |
| 4 | M/24 | Paranoid schizophrenia | Zuclopenthixol | 500 |
| | | | decanoate^{b} | |
| 5 | M/22 | Unspecified type | Zuclopenthixol | 100 |
| | | | decanoate^{b} | |
| 6 | M/32 | Paranoid schizophrenia | Zuclopenthixol | 200 |
| | | | decanoate^{b} | |

^{a}Daily per OS
^{b}Monthly IM

Fluvoxamine (100 mg/day) was added in an open study format to the antipsychotic treatment, which remained steady. Clinical state was assessed by psychiatrists using validated rating scales for negative (SANS) and positive (SAPS) symptoms (Silver et al., 2003) prior to fluvoxamine treatment and then weekly until the end of the trial period. Total SANS scores and summary scores for effective blunting, alogia, anhedonia and abolition factors were the outcome measures. Extrapyramidal symptoms were assessed with the Simpson-Angus Scale (SAS) for extrapyramidal side effects (Simpson and Angus, 1970). Blood samples were taken at baseline (day 0), before addition of fluvoxamine, after 3 weeks of combined treatment, a time when the initial clinical effect is expected to be seen (Silver et al., 2003), and after 6 weeks of the combined treatment.

2. Isolation of Peripheral Mononuclear Cells (PMC) from Blood

Blood samples (40 ml) from patients were collected in EDTA-coated tubes in order to prevent coagulation, and transported in ice to the laboratory for further processing. Each 15 ml of blood was completed to 40 ml with phosphate buffered saline (PBS) and gently mixed. PMC were isolated according to Ficoll protocol (Sigma, USA). In short, after addition of Ficoll reagent, the tubes were centrifuged (400 g, 30 min), and the interphase was transferred to a new tube, mixed with 100 ml PBS and centrifuged (400 g, 10 min). The supernatant was discarded and the pellet containing the PMC was washed 3 times with PBS (400 g, 10 min). TriReagent (Sigma, USA) was added to the pellet and left on ice for 15 min.

3. Extraction of Total RNA from Peripheral Mononuclear Cells (PMC)

200 ml chloroform was added to 1 ml of sample in TriReagent and the suspension was centrifuged (12,000 g, 20 min, 4° C.). After precipitation with isopropanol, the RNA pellet was washed twice with 70% ethanol (7,500 g, 10 min), followed by one wash with 96% ethanol (12,000 g, 10 min) and resuspended in diethylpyrocarbonate (DEPC)-treated water. RNA sample concentrations were determined by UV spectrophotometry at 260 nm (average OD260/2801.9-2). RNA purity was determined by the 260/230 and 260/280 ratios. RNA integrity was confirmed using gel (1.2% agarose) electrophoresis by direct visualization of 18S and 28S rRNA bands and densitometric analysis.

4. cDNA Array Analyses

Two types of cDNA expression membranes were employed. First, the gene expression profiles were examined using Atlas human 1.2 cDNA expression arrays II, including 1176 genes, according to the manufacturer's protocol (Clontech, Palo Alto, Calif., USA). A probe was generated for each one of the patients examined. Following the binding of the probe to the membranes, they were exposed to phosphor screen (BAS MP-2040 image plate, Fuji Inc., Tokyo, Japan) and the radioactive signals were detected with FLA-2000 scanner (Fuji Inc.). Quantitation and analysis of the radioactive signals were done using AtlasImage™ 2.01 software (Clontech). After global normalization, changes with log² ratio greater than 2SD of the mean were considered as significant (Nadon and Shoemaker, 2002).

In the next step, gene expression profiles were examined with GEArray™ cDNA membranes, consisting of genes related to G protein-coupled receptor (GPCR) pathways (Q series human G protein-coupled receptor gene array II, SuperArray, Bethesda, Md., USA). Each membrane consists of 100 human cDNAs fragments associated with GPCR family, including receptors for dopamine (DA), serotonin (5-HT), acetylcholine and epinephrine; receptors for chemokines; GPCR kinases; Mitogen activated protein kinases; and regulators of G-protein signaling (RGS). Hybridization array analysis was performed according to the manufacturer's (SuperArray, Bethesda, Md.) protocol. Total RNA from each patient was reverse transcribed with biotinylated dUTP (Roche Diagnostics, Mannheim, Germany) and a gene specific primer mix (SuperArray, Bethesda, Md.). The probes were hybridized to the membrane and the array image was recorded using X-ray film. Quantitation of the results and analysis was accomplished using the manufacturer Software Package (SuperArray, Bethesda, Md.). The row values from 4 spotted replicates of a gene were averaged, normalized to the median of all intensity values on array and compared to control values, thereby assessing the relative expression level of a given mRNA.

### 5. Real-Time RT-PCR

Two μg of total RNA were denatured and reverse transcribed using random hexanucleotides (0.5 μg/μl) as previously described (Chertkow et al., 2006). Real-time quantitative assessment was performed using LightCycler with FastStart DNA Master SYBR Green I ready-to use PCR mix kits according to the manufacture's protocol (Roche Diagnostics, Mannheim, Germany). Forty ng cDNA was amplified per sample. Each experimental set included one reaction with water as template to control for cross contamination. Amplified products were visualized on 1.5% agarose gel. The sequences of the primers, the experimental conditions and the melting temperature of the products are described in Table 2 hereinafter. The results were analyzed in real-time on the provided program of the LightCycler and normalized against a reference gene in order to correct sample-to-sample variation. Five potential reference genes for the human samples were considered: glyceraldehydes-3-phosphate dehydrogenase (GAPDH), gene encoding for peptidylprolyl isomerase B (cyclophin B, PPIB), β-actin, phosphomannomutase (PPMM) and 18S ribosomal RNA. Expression stability of these genes was determined in our samples with ‘Normfinder’ Excel applet (Andersen et al., 2004); PPIB was chosen as the reference gene for normalization. The normalized data was compared to control values to assess the relative expression level of a given mRNA.

### 6. Statistical Analysis

The biochemical data were analyzed by one way analysis of variance (ANOVA) followed by Dunnett's test. For each gene, differences between expression after 3 or 6 weeks of treatment ad baseline, were considered significant if they reached a level of significance of p<0.05. Clinical response was assessed with repeated measure ANOVA.

TABLE 2
Primer sequences and conditions for quantitative real-time RT-PCR

Conditions (° C., seconds)

| | | | | | | |
|---|---|---|---|---|---|---|
| Gene | ID NO. | Oligonucleotide sequence (5′-3′) | Dn | An | El | Ac |

| | | | | | | |
|---|---|---|---|---|---|---|
| 18S | 1 | F5′-GTTGGTGGAGCGATTTGTCT-3′ | 95(15) | 65(10) | 72(7) | 82/89 |
| | 2 | R5′-CGCTGAGCCAGTCAGTGTAG-3′ | | | | |
| β-actin | 3 | F5′-ACTGGAACGGTGAAGGTGAC-3′ | 95(15) | 65(10) | 72(10) | 85/86 |
| | 4 | R5′-GTGGACTTGGGAGAGGACTG-3′ | | | | |
| GAPDH | 5 | F5′-GCTGAGTACGTCGTGG-3′ | 95(15) | 65(10) | 72(10) | 85/88 |
| | 6 | R5′-GTGCTAAGCAGTTGGTG-3′ | | | | |
| PPIB | 7 | F5′-GCATCTACGGTGAGCG-3′ | 95(15) | 65(10) | 72(10) | 85/89 |
| | 8 | R5′-AGGGGTTTATCCCGGC-3′ | | | | |
| | 9 | F5′-AAGAGCATCTACGGTG-3′ | | | | |
| | 10 | R5′-GTTTATCCCGGCTGTC-3′ | | | | |
| PPMM | 11 | F5′-AAGCGTGGAACCTTCATCGA-3′ | 95(15) | 65(10) | 72(9) | 85/87 |
| | 12 | R5′-TCCCGGATCTTCTCTTTCTTGTC-3′ | | | | |
| IL8R | 13 | F5′-TGGGTTTTGGGGGGACG-3′ | 95(15) | 69(10) | 72(10) | 85/87 |
| | 14 | R5′-TGTCAGATTCGGGGCTC-3′ | | | | |
| CCR7 | 15 | F5′-ACTCCATCATTTGTTTCGTG-3′ | 95(15) | 69(10) | 72(10) | 85/90 |
| | 16 | R5′-TAGTATCCAGATGCCCACAC-3′ | | | | |
| CCR1 | 17 | F5′-ACCTGCAGCCTTCACTTTCCTCAC-3′ | 95(15) | 69(10) | 72(10) | 85/85 |
| | 18 | R5′-GGCGATCACCTCCGTCACTTG-3′ | | | | |
| RGS7 | 19 | F5′-CCTTCTAACCCATGGCTGTC-3′ | 95(15) | 69(10) | 72(10) | 85/86 |
| | 20 | R5′-TTTTTCAGGTCCTCCACTGC-3′ | | | | |
| GABA_{A}β2 | 21 | F5′-CGCATATTCTTCCCAGTGGT-3′ | 95(15) | 65(10) | 72(10) | 82/89 |
| | 22 | R5′-GCGTCACTTTTGTCCTGGAT 3′ | | | | |
| PKCβ2 | 23 | F5′-AAATTGCCATCGGTCTGTTC-3′ | 95(15) | 65(10) | 72(10) | 85/89 |
| | 24 | R5′-CCCATAGGGCTGATAAGCAA-3′ | | | | |

Dn - Denaturation, An - Annealing, El - Elongation, Ac - Acquisition T/Tm, F - Forward, R - Reverse; All templates were initially denatured for 10 min at 95° C. Amplification was done for 35 cycles. Melting curve analysis was done by continues acquisition from 65° C. to 95° C. with temperature transition rate of 0.1° C./sec. RGS - regulator of G-protein; IL8R - interleukin receptor 8A; CCR - Chemokine (C-C motif) receptor, GABA - gamma aminobutyric acid; PKC - proteinkinase C.

### Example 1

### Gene Expression Profiles in PMCS of Schizophrenic Patients During Combined Antipsychotic-Antidepressant Treatment

In this study, gene expression changes in the peripheral mononuclear cells (PMCs) of schizophrenic patients during 6 weeks of combined antipsychotic-antidepressant treatment were examined. In particular, patients suffering from negative symptoms despite constant antipsychotic treatment for at least 4 weeks were co-treated with fluvoxamine as described in Materials and Methods. Blood samples were taken and clinical state was assessed at baseline, before addition of fluvoxamine, and after 3 and 6 weeks of combined treatment, so that each patient served as his own control.

Gene expression changes with treatment were determined per patient, relative to his own baseline mRNA level. The within-subject comparison reduces confounds due to inter individual variability and illness heterogeneity factors and places the focus on treatment-related changes. Table 3 hereinafter shows the results of a preliminary screening study in 4 patients (global cDNA expression array, Clontech, Palo Alto, Calif., USA). Transcript changes homologous in at least 3 out of the 4 subjects were found in genes associated with G-protein signaling cascades, including G-proteins (g(i), α2 subunit and g(q) α subunit), receptor of activated protein kinase C1 (RACK1), 1,4,5-trisphosphate 3-kinase, and phosphatidylinositol transfer protein alpha isoform (PI-TPα).

Based on the initial data described above and on the known interactions of neuroleptics with dopamine and serotonin receptors, we then examined GPCR-related genes, using a customized cDNA array, and found that 10% of the genes showed homologous changes in at least 4 out of the 6 subjects. As shown in Table 4, summarizing the relative changes in PMC gene expression after 3 and 6 weeks of the combined treatment, with respect to the chemokine receptors family, a decrease in the expression level of chemokine (C-C motif) receptor 1 (CCR1), chemokine (C-C motif) receptor-like 1 (CCRL1), chemokine-like receptor 1 (CMKLR1) and interleukin 8 receptor alpha (IL8Rα) was observed in at least 4 out of the 6 subjects after 3 weeks or more of the combined treatment, whereas an increase was observed in the expression level of CCR5. As specifically noted, the expression level of both chemokine (C-C motif) receptor 7 (CCR7) and CCRL1 in week 6 was increased compared with their expression level in week 3. Furthermore, reduced expression level was noted in transcripts encoding for 5-HT receptors (5-HT_{2A} and 5-HR₇) And for regulator of G-protein signaling 7 (RGS7), after 3 weeks or more of the combined treatment.

TABLE 3
mRNA expression changes* in PMC from schizophrenic patients
following fluvoxamine augmentation treatment

| | | Patient | | | |
|---|---|---|---|---|---|
| Gene code | Gene | 1 | 2 | 3 | 4 |
| X04828 | G-protein g(i), α-2 subunit | d | d | d | d |
| U43083 | G-protein g(q), α subunit (gnaq or gaq) | d | d | d | nc |
| M24194 | Receptor of activated protein kinase C1 (RACK1) | d | d | d | d |
| X57206 | 1,4,5-trisphosphate 3-kinase | d | d | d | nc |
| M73704 | Phosphatidylinositol transfer protein α isoform (PI-TP-α) | d | nc | d | d |
| Y09689 | Neurogranin (NRGN); RC3 | d | d | d | nc |
| X14046 | Leukocyte CD37 antigen | d | d | d | nc |
| AF012629 | Antagonist decoy receptor for TRAIL/APO2L (TRID) | d | d | nc | d |
| M21130 | Neutrophil defensins 1, 2 and 3 precursor (hnp) | d | nc | d | d |
| | (defensin, α 1) | | | | |
| X75918 | Nuclear receptor-related 1 | d | nc | d | d |
| M27507 | Acid beta-galactosidase; GLB1 | d | nc | d | d |
| D29992 | Tissue factor pathway inhibitor 2 | d | nc | d | d |
| U47742 | Zinc finger protein moz = | nc | d | d | d |
| | monocytic leukemia zinc finger protein | | | | |
| M26880 | Ubiquitin | d | d | d | d |
| X00351 | Cytoplasmic beta-actin (ACTB) | d | d | d | d |
| X56932 | 60S ribosomal protein L13A (RPL13A) | d | d | d | d |
| U14971 | 40S ribosomal protein S9 | d | d | d | d |
| X01677 | Liver glyceraldehyde 3-phosphate dehydrogenase | nc | d | d | d |
| | (GAPDH) | | | | |

*nc: no change; d: down.

TABLE 4
Gene expression changes* in PMC from schizophrenic patients following
fluvoxamine augmentation treatment

| | | Patient | | | | | | | | | | | |
|---|---|---|---|---|---|---|---|---|---|---|---|---|---|
| | | I | | II | | III | | IV | | V | | VI | |
| | | Week | | | | | | | | | | | |
| Gene code | Gene | 3 | 6 | 3 | 6 | 3 | 6 | 3 | 6 | 3 | 6 | 3 | 6 |
| D10925 | CCR1 | nd | U | D | D | nc | D | D | D | nd | nd | D | D |
| X91492 | CCR5 | nd | nd | nd | D | U | nd | U | nc | U | U | U | nc |
| L31581 | CCR7 | U | U | D | nc | D | D | nc | U | nc | U | D | nc |
| AF110640 | CCRL1 | nc | D | D | nc | D | D | nc | U | nc | U | D | nc |
| U79527 | CRL1 | D | D | D | D | nc | D | D | U | D | D | nc | nc |
| L19591 | IL8Rα | U | U | D | nc | D | D | U | D | D | D | nc | D |
| BC022009 | RGS7 | D | D | nc | D | nc | D | U | nc | D | D | nc | nc |
| X57830 | 5-HT_{2A} | D | D | nc | D | D | D | D | D | nd | nd | D | nc |
| L21195 | 5-HT₇ | nd | nd | D | nc | nc | D | D | D | D | D | D | D |

*mRNA expression levels at weeks 3 and 6 were normalized, i.e. divided by week 0;
D—down regulation (<0.7);
U—up regulation (>1.3);
nc—No change;
nd—Not detected.

### Example 2

### Real Time RT-PCR Analysis of Selected mRNAs in the PMC from Schizophrenic Patients Treated with Antipsychotic Plus Fluvoxamine

In this study, the significant gene expression changes in the PMC of schizophrenic patients, observed in the customized array and shown in Example 1, were verified by real-time RT-PCR. In order to obtain reliable normalization specific for our tissue and experimental design, expression stabilities of five potential reference genes were examined. These genes were selected based on the literature and included GAPDH, PPIB, β-actin, PPMM and 18S rRNA (Malarstig et al., 2003; Bas et al., 2004; Garcia-Vallejo et al., 2004; Pachot et al., 2004). The expression level of each candidate was assessed in all samples. PPIB, PPMM and 18S showed the most stable expression in our population, and based on analysis in ‘Normfinder’ software, PPIB was chosen as the normalization gene for the real-time RT-PCR assays.

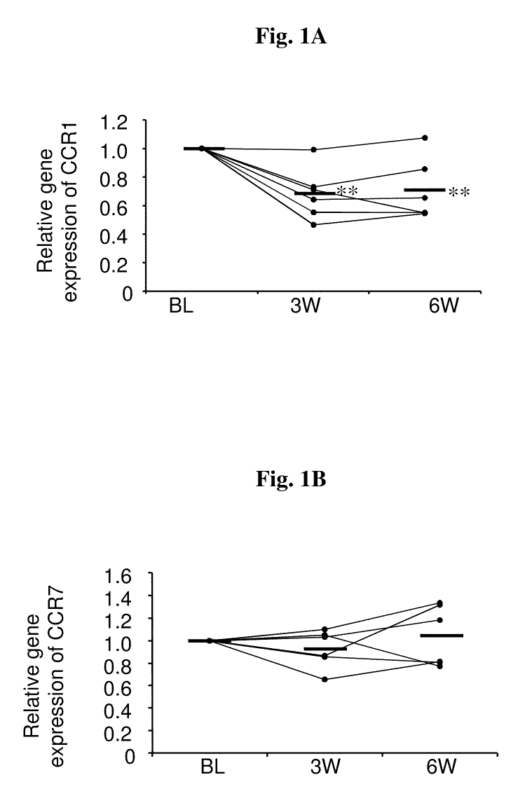

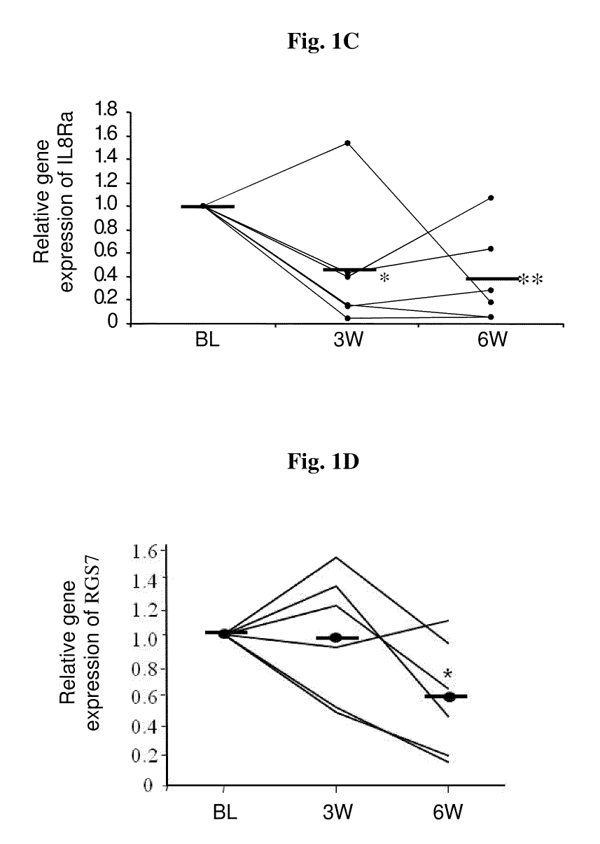

The genes examined by real-time RT-PCR were IL8Rα, CCR1, CCR7 and RGS7. As shown in FIGS. 1A-1D, IL8Rα mRNA expression was reduced significantly after 3 and 6 weeks of fluvoxamine add-on treatment compared with the initial level of this gene product, confirming the array data. Likewise, CCR1 mRNA expression was reduced at both time points, in 5 out of 6 patients. CCR7 did not show a consistent trend among the patients. RGS7 was significantly reduced after 6 weeks.

### Example 3

### Observation of Clinical Response in Patients Following Augmentation-Treatment

As shown in Table 5 and Table 6 hereinbelow, following augmentation-treatment, significant changes were observed with mean rating scales for negative (SANS) total score (p<0.001); affective blunting (p<0.01); alogia (p<0.01) and a trend for anhedonia (p=0.30) and avolition (p=0.75) factors. Extra pyramidal side effects were absent in all, except one patient, and did not change significantly with augmentation treatment. There was no significant change in rating scales for positive (SAPS) score.

TABLE 5
Total SANS and SAPS scores in schizophrenic patients following
fluvoxamine augmentation treatment

| | SANS total | | | SAPS total | | |
|---|---|---|---|---|---|---|
| Patient | BL | 3 W | 6 W | BL | 3 W | 6 W |
| I | 111 | 104 | 103 | 14 | 12 | 12 |
| II | 93 | 91 | 86 | 11 | 11 | 10 |
| III | 102 | 97 | 98 | 13 | 12 | 12 |
| IV | 82 | 71 | 63 | 9 | 9 | 9 |
| V | 52 | 49 | 46 | 6 | 7 | 7 |
| VI | 73 | 69 | 60 | 8 | 7 | 6 |

* BL: at baseline (day 0); 3 W and 6 W: after 3 and 6 weeks, respectively, of augmentation treatment

TABLE 6
Symptom scores in schizophrenic patients following fluvoxamine
augmentation treatment

| | | | | | | | | | | | | | Extra- | | |
|---|---|---|---|---|---|---|---|---|---|---|---|---|---|---|---|
| | Affective | | | | | | | | | | | | pyramidal | | |
| | blunting | | | Alogia | | | Anhedonia | | | Avolition | | | side effects | | |
| Patient | BL | 3 W | 6 W | BL | 3 W | 6 W | BL | 3 W | 6 W | BL | 3 W | 6 W | BL | 3 W | 6 W |
| I | 28 | 27 | 67 | 60 | 54 | 26 | 19 | 17 | 17 | 19 | 19 | 19 | 0 | 0 | 0 |
| II | 24 | 23 | 91 | 89 | 89 | 21 | 14 | 14 | 14 | 16 | 16 | 16 | 0 | 0 | 0 |
| III | 26 | 26 | 100 | 100 | 104 | 26 | 16 | 16 | 14 | 18 | 18 | 18 | 0 | 0 | 0 |
| IV | 23 | 19 | 30 | 24 | 24 | 16 | 14 | 11 | 8 | 13 | 11 | 11 | 6 | 6 | 4 |
| V | 11 | 9 | 22 | 21 | 21 | 9 | 11 | 11 | 9 | 9 | 8 | 8 | 0 | 0 | 0 |
| VI | 19 | 18 | 14 | 14 | 13 | 15 | 14 | 14 | 12 | 10 | 8 | 8 | 0 | 0 | 0 |

* BL: at baseline (day 0); 3 W and 6 W: after 3 and 6 weeks, respectively, of augmentation treatment

### Example 4

### Real Time RT-PCR Analysis of Selected mRNAs in the PMC from Schizophrenic Patients Treated with Antipsychotic Plus Fluvoxamine

In this study, which is similar to that described in Example 2 hereinabove, the antidepressant fluvoxamine was added to the constant antipsychotic treatment of 8 patients, suffering from chronic schizophrenia with persistent negative symptoms. Total RNA, isolated from the PMC of these patients at baseline, 1, 3 and 6 weeks of dual treatment was reverse transcribed. Based on our previous animal study (Chertkow et al., 2006), cDNA was amplified in quantitative real-time PCR using suitable primers for GABA_{A}β2 and PKCβ2. PPIB was chosen as the normalization gene for the real-time RT-PCR assays.

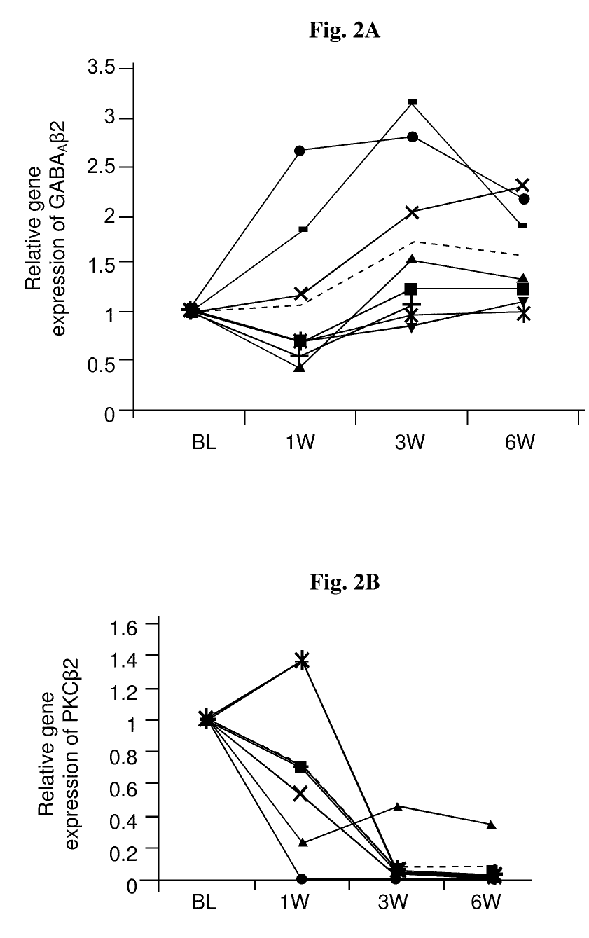

As shown in FIGS. 2A-2B, the average GABA_{A}β2 expression measured in the 8 patients participated in this study, after 3-6 weeks of the combined treatment, increased by about 40-50% compared with day 0, while the average PKCβ2 expression measured in these patients, decreased by about 30%, compared with day 0, after one week of the combined treatment, and by about 90% or more, compared with day 0, after 3-6 weeks of the combined treatment.

### REFERENCES

Andersen C. L., Jensen J. L., Ørntoft T. F., Normalization of real-time quantitative reverse transcription-PCR data: a model-based variance estimation approach to identify genes suited for normalization, applied to bladder and colon cancer data sets, Cancer Res, 2004, 64, 5245-5250
Avissar S., Roitman G., Schreiber G., Differential effects of the antipsychotics haloperidol and clozapine on G protein measures in mononuclear leukocytes of patients with schizophrenia, Cell Mol Neurobiol, 2001, 21, 799-811
Barak V., Barak Y., Levine J., Nisman B., Roisman I., Changes in interleukin-1 beta and soluble interleukin-2 receptor levels in CSF and serum of schizophrenic patients, J Basic Clin Physiol Pharmacol, 1995, 6, 61-69
Bas A., Forsberg G., Hammarstrom S., Hammarstrom M. L., Utility of the housekeeping genes 18S rRNA, beta-actin and glyceraldehyde-3-phosphatedehydrogenase for normalization in real-time quantitative reverse transcriptase-polymerase chain reaction analysis of gene expression in human T Lymphocytes, Scand J Immunol, 2004, 59, 566-573
Benzing T., Brandes R., Sellin L., Schermer B., Lecker S., Walz G., Kim E., Upregulation of RGS7 may contribute to tumor necrosis factor-induced changes in central nervous function, Nat Med, 1999, 5, 913-918
Bowden N. A., Weidenhofer J., Scott R. J., Schall U., Todd J., Michie P. T., Tooney, P. A., Preliminary investigation of gene expression profiles in peripheral blood lymphocytes in schizophrenia, Schizophr Res, 2006, 82, 175-183
Bowden N. A., Scott R. J., Tooney P. A., Altered expression of regulator of G-protein signalling 4 (RGS4) mRNA in the superior temporal gyrus in schizophrenia, Schizophr Res, 2007, 89, 165-168
Broadbelt K., Ramprasaud A., Jones L. B., Evidence of altered neurogranin immunoreactivity in areas 9 and 32 of schizophrenic prefrontal cortex, Schizophr Res, 2006, 87, 6-14
Brown A. S., Hooton J., Schaefer C. A., Zhang H., Petkova E., Babulas V., Perrin M., Gorman J. M., Susser E. S., Elevated maternal interleukin-8 levels and risk of schizophrenia in adult offspring, Am J Psychiatry, 2004, 161, 889-895
Chertkow Y., Weinreb O., Youdim M. B., Silver H., The effect of chronic co-administration of fluvoxamine and haloperidol compared to clozapine on the GABA system in the rat frontal cortex, Int J. Neuropsychopharmacol., 2006, 9, 287-296
Chertkow Y., Weinreb O., Youdim M. B. H., Silver H, Gene expression changes in peripheral mononuclear cells from schizophrenic patients treated with a combination of antipsychotic with fluvoxamine, Prog. Neu. Psych. Bio. Psych., 2007, 31, 1356-1362
Dean B., Hussain T., Hayes W., Scarr E., Kitsoulis S., Hill C., Opeskin K., Copolov D. L., Changes in serotonin₂A and GABA_{A} receptors in schizophrenia: studies on the human dorsolateral prefrontal cortex, J Neurochem, 1999, 72, 1593-1599
Erbagci A. B., Herken H., Koyluoglu O., Yilmaz N., Tarakcioglu M., Serum IL-1beta, Sil-2R, IL-6, IL-8 and TNF-alpha in schizophrenic patients, relation with symptomatology and responsiveness to risperidone treatment, Mediators Inflamm, 2001, 10, 109-115
Garcia-Vallejo J. J., Van het H of B., Robben J., Van Wijk J. A. E., Van Die I., Joziasse D. H., Van Dijk W., Approach for defining endogenous reference genes in gene expression experiments, Anal Biochem, 2004, 329, 293-299
Ghavami A., Hunt R. A., Olsen M. A., Zhang J., Smith D. L., Kalgaonkar S., Rahman Z., Young K. H., Differential effects of regulator of G protein signaling (RGS) proteins on serotonin 5-HT1A, 5-HT2A, and dopamine D2 receptor-mediated signaling and adenylyl cyclase activity, Cell Signal, 2004, 16, 711-721
Gladkevich A., Kauffman H. F., Korf J., Lymphocytes as a neural probe: potential for studying psychiatric disorders, Prog Neuropsychopharmacol Biol Psychiatry, 2004, 28, 559-576
Goff D. C., Midha K. K., Sarid-Segal O., Hubbard J. W., Amico E., A placebo-controlled trial of fluoxetine added to neuroleptic in patients with schizophrenia, Psychopharmacology (Ber), 1995, 117, 417-423
Gold S. J., Heifets B. D., Pudiak C. M., Potts B. W., Nestler E. J., Regulation of regulators of G protein signaling mRNA expression in rat brain by acute and chronic electroconvulsive seizures, J Neurochem, 2002, 82, 828-838
Grimaldi B., Fillion G., 5-HT-moduline controls serotonergic activity: implication in neuroimmune reciprocal regulation mechanisms, Prog Neurobiol, 2000, 60, 1-12
Huang X. F., Han M., Huang X., Zavitsanou K., Deng C., Olanzapine differentially affects 5-HT_{2A} and 5-HT₂c receptor mRNA expression in the rat brain, Behav Brain Res, 2006, 171, 355-362
Ilani T., Ben-Shachar D., Strous R. D., Mazor M., Sheinkman A., Kotler M., Fuchs S., A peripheral marker for schizophrenia: increased levels of D3 dopamine receptor mRNA in blood lymphocytes, Proc Natl Acad Sci USA, 2001, 98, 625-628
Kim E., Arnould T., Sellin L., Benzing T., Comella N., Kocher O., Tsiokas L., Sukhatme V., Walz G., Interaction between RGS7 and polycystin, Proc Natl Acad Sci USA, 1999, 96, 6371-6376
Kim Y. K., Kim L., Lee M. S., Relationships between interleukins, neurotransmitters and psychopathology in drug-free male schizophrenics, Schizophr Res, 2000, 44, 165-175
Kronfol Z., Remick D. G., Cytokines and the brain: implications for clinical psychiatry, Am J Psychiatry, 2000, 157, 683-694
Larminie C., Murdock P., Walhin J. P., Duckworth M., Blumer K. J., Scheideler M. A., Garnier M., Selective expression of regulators of G-protein signaling (RGS) in the human central nervous system, Brain Res Mol Brain Res, 2004, 122, 24-34
Lieberman J., Stroup T. S., McEvoy J., Swartz M., Rosenheck R., Perkins D. O., Keefe R. S. E., Davis S. M., Davis C. E., Lebowitz B. D., Severe J., Hsiao J. K., Effectiveness of antipsychotic drugs in patients with chronic schizophrenia, N Engl J Med., 2005, 353, 1209-1223
Liew C. C., Ma J., Tang H. C., Zheng R., Dempsey A. A., The peripheral blood transcriptome dynamically reflects system wide biology: a potential diagnostic tool, J Lab Clin Med, 2006, 147, 126-132
Maes M., Bocchio Chiavetto L., Bignotti S., Battisa Tura G. J., Pioli R., Boin F., Kenis G., Bosmans E., de Jongh R., Altamura C. A., Increased serum interleukin-8 and interleukin-10 in schizophrenic patients resistant to treatment with neuroleptics and the stimulatory effects of clozapine on serum leukemia inhibitory factor receptor, Schizophr Res, 2002, 54, 281-291
Mälarstig A., Tenno T., Jossan S., Åberg M., Siegbahn A., A quantitative real-time PCR method for tissue factor mRNA, Thromb Res, 2003, 112, 175-183
Mirnics K., Middleton F. A., Stanwood G. D., Lewis D. A., Levitt P., Disease-specific changes in regulator of G-protein signaling 4 (RGS4) expression in schizophrenia, Mol Psychiatry, 2001, 6, 293-301
Muller N., Riedel M., Ackenheil M., Schwarz M. J., The role of immune function in schizophrenia: an overview, Eur Arch Psychiatry Clin Neurosci, 1999, 249, 62-68
Murphy B. P., Chung Y. C., Park T. W., McGorry P. D., Pharmacological treatment of primary negative symptoms in schizophrenia: a systematic review, Schizophr Res., 2006, 88, 5-25
Nadon R., Shoemaker J., Statistical issues with microarrays: processing and analysis, Trends Gent, 2002, 18, 265-271
Opeskin K., Dean B., Pavey G., Hill C., Keks N., Copolov D., Neither protein kinase C nor adenylate cyclase are altered in the striatum from subjects with schizophrenia, Schizophr Res, 1996, 22, 159-164
Pachot A., Blond J. L., Mougin B., Miossec P., Peptidylpropyl isomerase B (PPIB): a suitable reference gene for mRNA quantification in peripheral whole blood, J Biotechnol, 2004, 114, 121-124
Rothermundt M., Arolt V., Bayer T. A., Review of immunological and immunopathological findings in schizophrenia, Brain Behav Immun, 2001, 15, 319-339
Silver H., Fluvoxamine as an adjunctive agent in schizophrenia, CNS Drug Rev, 2001, 7, 283-304
Silver H., Nassar A., Fluvoxamine improves negative symptoms in treated chronic schizophrenia: an add-on double-blind, placebo-controlled study, Biol Psychiatry, 1992, 31, 698-704
Silver H., Kushnir M., Kaplan A., Fluvoxamine augmentation in clozapine-resistant schizophrenia: an open pilot study, Biol Psychiatry, 1996, 40, 671-674
Silver H., Youdim M. B., MAO-A and MAO-B activities in rat striatum, frontal cortex and liver are unaltered after long-term treatment with fluvoxamine and desipramine, Eur Neuropsychopharmacol., 2000, 10, 125-128
Silver H., Nassar A., Aharon N., Kaplan A., The onset and time course of response of negative symptoms to add-on fluvoxamine treatment, Int Clin Psychopharmacol, 2003, 18, 87-92
Silver H., Shmugliakov N., Augmentation with fluvoxamine but not maprotiline improves negative symptoms in treated schizophrenia: evidence for a specific serotonergic effect from a double-blind study, J Clin Psychopharmacol., 1998, 18, 208-211

Simpson G. M., Angus J. W. S., A rating scale for extrapyramidal side effects. Acta Psychiatr Scand, 1970, 212, 11-19
Spina E., De Domenico P., Ruello C., Longobardo N., Gitto C., Ancione M., Di Rosa A. E., Caputi A. P., Adjunctive fluoxetine in the treatment of negative symptoms in chronic schizophrenic patients, Int Clin Psychopharmacol., 1994, 9, 281-285
Sullivan P. F., Fan C., Perou C. M., Evaluating the comparability of gene expression in blood and brain, Am J Med Genet B Neuropsychiatr Genet, 2006, 141, 261-268
Tang Y., Gilbert D. L., Glauser T. A., Hershey A. D., Sharp F. R., Blood gene expression profiling of neurologic diseases: a pilot microarray study, Arch Neurol, 2005, 62, 210-215
Tardito D., Maina G., Tura G. B., Bogetto F., Pioli R., Ravizza L., Racagni G., Perez J., The cAMP-dependent protein kinase substrate Rap1 in platelets from patients with obsessive compulsive disorder or schizophrenia, Eur Neuropsychopharmacol, 2001, 11, 221-225
Toyooka K., Watanabe Y., Iritani S., Shimizu E., Iyo M., Nakamura R., Asama K., Makifuchi T., Kakita A., Takahashi H., Someya T., Nawa H., A decrease in interleukin-1 receptor antagonist expression in the prefrontal cortex of schizophrenic patients, Neurosci Res, 2003, 46, 299-307
van Hes R., Smid P., Stroomer C. N. J., Tipker K., Tulp M. T. M., van der Heyden J. A. M., McCreary A. C., Hesselink M. B., Kruse C. G., SLV310, a novel, potential antipsychotic, combining potent dopamine D₂ receptor antagonism with serotonin reuptake inhibition, Bioorg. Med. Chem. Lett., 2003, 13, 405-408
Wilson C. J., Finch C. E., Cohen H. J., Cytokines and cognition—the case for a headto-toe inflammatory paradigm, J Am Geriatr Soc, 2002, 50, 2041-2056
Zhang X. Y., Zhou D. F., Zhang P. Y., Wu G. Y., Cao L. Y., Shen Y. C., Elevated interleukin-2, interleukin-6 and interleukin-8 serum levels in neuroleptic-free schizophrenia: association with psychopathology, Schizophr Res, 2002, 57, 247-258
Zhang X. Y., Zhou D. F., Cao L. Y., Zhang P. Y., Wu G. Y., Shen Y. C., Changes in serum interleukin-2, -6, and -8 levels before and during treatment with risperidone and haloperidol: relationship to outcome in schizophrenia, J Clin Psychiatry, 2004, 65, 940-947

1. A method for evaluating the pharmacological efficacy of a drug candidate in treatment of a psychiatric disease or disorder, said method comprising:
(i) administering to each individual in a group of patients having said psychiatric disease or disorder said drug candidate for a sufficient time period;
(ii) measuring expression levels of genes expressed in peripheral mononuclear cells (PMCs) in blood samples obtained from said patients at a first instant before the first administration of said drug candidate and at given second and third instants following the first administration of said drug candidate, thus obtaining a test gene expression profile expressing a representative relative level of each one of said genes at said second and third instants for said group of patients; and
(iii) comparing said test gene expression profile with either (a) a reference gene expression profile obtained as described in (ii) from a group of patients administered with a drug or drug combination effective against both positive and negative symptoms of psychiatric diseases or disorders, or (b) a predetermined reference gene expression profile expressing a representative relative level of each one of said genes at said second and third instants indicating an effective treatment against both positive and negative symptoms of psychiatric diseases or disorders,
wherein a significant similarity between said test gene expression profile and said reference gene expression profile or predetermined reference gene expression profile indicates that said drug candidate has a likelihood of being effective in treatment of said psychiatric disease or disorder.

2. The method of claim 1, wherein said drug combination effective against both positive and negative symptoms of psychiatric diseases or disorders is a combination of an antipsychotic agent and an antidepressant agent functioning pharmacologically as a selective serotonin reuptake inhibitor (SSRI).

3. The method of claim 2, wherein said antipsychotic agent is selected from the group consisting of risperidone, olanzapine, ziprasidone, clozapine, haloperidol, perphenazine, trifluperazine, amisulpride, chlorprothixene, thiothixene, flupentixol and zuclopenthixol, and said antidepressant agent is fluvoxamine or fluoxetine.

4. The method of claim 3, wherein said drug combination effective against both positive and negative symptoms of psychiatric diseases or disorders is a combination of haloperidol and fluvoxamine.

5. The method of claim 1, wherein said second and third instants are 2 to 4 and 5 to 7 weeks, respectively, following the first administration of said drug candidate.

6. The method of claim 5, wherein said second and third instants are about 3 and about 6 weeks, respectively, following the first administration of said drug candidate.

7. The method of claim 1, wherein said genes expressed in PMCs encode for G-protein-coupled receptors (GPCRs), proteins involved in primary metabolism, calcium signaling regulators, or cell signaling regulators.

8. The method of claim 7, wherein said GPCRs are selected from the group consisting of a chemokine receptor, a chemokine-like receptor, a regulator of G-protein signaling, a serotonin (5-hydroxytryptamine, 5-HT) receptor, guanine nucleotide-binding protein G(i) subunit alpha-2, guanine nucleotide-binding protein G(q) subunit alpha, receptor of activated protein kinase C 1 (RACK1) and gamma aminobutyric acid (GABA)_{A}β2; said proteins involved in primary metabolism are selected from the group consisting of nuclear receptor-related 1 (NURR1), phosphatidylinositol transfer protein alpha isoform (PI-TP-alpha), acid beta-galactosidase (GLB-1) and ubiquitin; said calcium signaling regulators are 1,4,5-trisphosphate 3-kinase or neurogranin (NRGN); and said cell signaling regulators are selected from the group consisting of protein kinase C (PKC)β2, extracellular signal-regulated kinase 1 (ERK1) and ERK2.

9. The method of claim 8, wherein said chemokine receptor is selected from the group consisting of chemokine (C-C motif) receptor 1-10 (CCR1-CCR10), chemokine (C-C motif) receptor-like 1 (CCRL1) and interleukin 8 receptor alpha (IL8Rα); said chemokine-like receptor is chemokine-like receptor 1 (CMKLR1); said regulator of G-protein signaling is regulator of G-protein signaling 2, 4 or 7 (RGS2, RGS4 or RGS7, respectively); and said serotonin receptor is 5-HT_{2A}, 5HT_{3A}, 5HT_{3B} or 5HT₇.

10. The method of claim 7, wherein said genes expressed in PMCs encode for the G-protein-coupled receptors CCR1, CCR5, CCR7, CCRL1, IL8Rα, CMKLR1, RGS7, 5-HT_{2A}, 5-HT₇ and GABA_{A}β2, and for the cell signaling regulators PKCβ2.

11. The method of claim 1, wherein said genes expressed in PMCs encode for CCR1, CCR5, CCR7, CCRL1, IL8Rα, CMKLR1, RGS7, 5-HT_{2A}, 5-HT₇, GABA_{A}β2 and PKCβ2; said second and third instants are about 3 and about 6 weeks, respectively, following the first administration of said drug candidate; and said reference gene expression profile or predetermined reference gene expression profile shows a decrease in the CCR1, CCRL1, CMKLR1, IL8Rα, RGS7, 5-HT_{2A}, 5-HT₇ and PKCβ2 gene expression levels at said second or third instant relative to said first instant; an increase in the CCR5 and GABA_{A}β2 gene expression levels at said second or third instant relative to said first instant; and an increase in CCR7 and CCRL1 gene expression levels at said third instant relative to said second instant.

12. The method of claim 1, wherein said psychiatric disease or disorder is selected from the group consisting of schizophrenia, obsessive-compulsive disorder (OCD), major depression, bipolar disorder or dementia that may be accompanied or complicated by affective disorder or aggression.

13. The method of claim 12, wherein said psychiatric disease or disorder is schizophrenia.

14. A method for evaluating the pharmacological efficacy of a drug candidate in treatment of schizophrenia, said method comprising:
(i) administering to each individual in a group of patients having schizoprenia said drug candidate for a sufficient time period;
(ii) measuring expression levels of the genes CCR1, CCR5, CCR7, CCRL1, CMKLR1, IL8Rα, RGS7, 5-HT_{2A}, 5-HT₇, GABA_{A}β2 and PKCβ2, in peripheral mononuclear cells (PMCs) in blood samples obtained from said patients at a first instant before the first administration of said drug candidate and at second and third instants about 3 and 6 weeks, respectively, following the first administration of said drug candidate, thus obtaining a test gene expression profile expressing a representative relative level of each one of said genes at said second and third instants for said group of patients; and
(iii) analyzing said test gene expression profile,
wherein a decrease in the CCR1, CCRL1, CMKLR1, IL8Rα, RGS7, 5-HT_{2A}, 5-HT₇ and PKCβ2 gene expression levels at said second or third instant relative to said first instant; together with an increase in the CCR5 and GABA_{A}β2 gene expression levels at said second or third instant relative to said first instant; and together with an increase in CCR7 and CCRL1 gene expression levels at said third instant relative to said second instant, indicate that said drug candidate has a likelihood of being effective in treatment of schizophrenia.

15. A method for predicting the efficacy of a drug or drug combination indicated for treatment of both positive and negative symptoms of psychiatric diseases or disorders in a patient having a psychiatric disease or disorder, said method comprising:
(i) administering to said patient said drug or drug combination for a sufficient time period;
(ii) measuring expression levels of genes expressed in peripheral mononuclear cells (PMCs) in blood samples obtained from said patient at a first instant before the first administration of said drug or drug combination and at given second and third instants following the first administration of said drug or drug combination, thus obtaining a test gene expression profile expressing a relative level of each one of said genes at said second and third instants for said patient; and
(iii) comparing said test gene expression profile with a predetermined reference gene expression profile expressing a representative relative level of each one of said genes at said second and third instants indicating an effective treatment against both positive and negative symptoms of psychiatric diseases or disorders,
wherein a significant similarity between said test gene expression profile and said predetermined reference gene expression profile indicates that said drug or drug combination has a likelihood of being effective in treatment of said patient.

16. The method of claim 15, wherein said second and third instants are 2 to 4 and 5 to 7 weeks, respectively, following the first administration of said drug or drug combination.

17. The method of claim 16, wherein said second and third instant are about 3 and about 6 weeks, respectively, following the first administration of said drug or drug combination.

18. The method of claim 15, wherein said genes expressed in PMCs encode for G-protein-coupled receptors (GPCRs), proteins involved in primary metabolism, calcium signaling regulators, or cell signaling regulators.

19. The method of claim 18, wherein said GPCRs are selected from the group consisting of a chemokine receptor, a chemokine-like receptor, a regulator of G-protein signaling, a serotonin (5-hydroxytryptamine, 5-HT) receptor, guanine nucleotide-binding protein G(i) subunit alpha-2, guanine nucleotide-binding protein G(q) subunit alpha, receptor of activated protein kinase C 1 (RACK1) and gamma aminobutyric acid (GABA)_{A}β2; said proteins involved in primary metabolism are selected from the group consisting of nuclear receptor-related 1 (NURR1), phosphatidylinositol transfer protein alpha isoform (PI-TP-alpha), acid beta-galactosidase (GLB-1) and ubiquitin; said calcium signaling regulators are 1,4,5-trisphosphate 3-kinase or neurogranin (NRGN); and said cell signaling regulators are selected from the group consisting of protein kinase C (PKC)β2, extracellular signal-regulated kinase 1 (ERK1) and ERK2.

20. The method of claim 19, wherein said chemokine receptor is selected from the group consisting of chemokine (C-C motif) receptor 1-10 (CCR1-CCR10), chemokine (C-C motif) receptor-like 1 (CCRL1) and interleukin 8 receptor alpha (IL8Rα); said chemokine-like receptor is chemokine-like receptor 1 (CMKLR1); said regulator of G-protein signaling is regulator of G-protein signaling 2, 4 or 7 (RGS2, RGS4 or RGS7, respectively); and said serotonin receptor is 5-HT_{2A}, 5HT_{3A}, 5HT_{3B} or 5HT₇.

21. The method of claim 18, wherein said genes expressed in PMCs encode for the G-protein-coupled receptors CCR1, CCR5, CCR7, CCRL1, IL8Rα, CMKLR1, RGS7, 5-HT_{2A}, 5-HT₇ and GABA_{A}β2, and for the cell signaling regulators PKCβ2.

22. The method of claim 15, wherein said genes expressed in PMCs encode for CCR1, CCR5, CCR7, CCRL1, IL8Rα, CMKLR1, RGS7, 5-HT_{2A}, 5-HT₇, GABA_{A}β2 and PKCβ2; said second and third instants are about 3 and about 6 weeks, respectively, following the first administration of said drug candidate; and said predetermined reference gene expression profile shows a decrease in the CCR1, CCRL1, CMKLR1, IL8Rα, RGS7, 5-HT_{2A}, 5-HT₇ and PKCβ2 gene expression levels at said second or third instant relative to said first instant; an increase in the CCR5 and GABA_{A}β2 gene expression levels at said second or third instant relative to said first instant; and an increase in CCR7 and CCRL1 gene expression levels at said third instant relative to said second instant.

23. The method of claim 15, wherein said psychiatric disease or disorder is selected from the group consisting of schizophrenia, obsessive-compulsive disorder (OCD), major depression, bipolar disorder or dementia that may be accompanied or complicated by affective disorder or aggression.

24. The method of claim 23, wherein said psychiatric disease or disorder is schizophrenia.

25. A method for predicting the efficacy of a drug or drug combination indicated for treatment of both positive and negative symptoms of psychiatric diseases or disorders in a patient having schizophrenia, said method comprising:
(i) administering to said patient said drug or drug combination for a sufficient time period;
(ii) measuring expression levels of the genes CCR1, CCR5, CCR7, CCRL1, CMKLR1, IL8Rα, RGS7, 5-HT_{2A}, 5-HT₇, GABA_{A}β2 and PKCβ2, in peripheral mononuclear cells (PMCs) in blood samples obtained from said patient at a first instant before the first administration of said drug or drug combination and at second and third instants about 3 and 6 weeks, respectively, following the first administration of said drug or drug combination, thus obtaining a test gene expression profile expressing a relative level of each one of said genes at said second and third instants for said patient; and
(iii) analyzing said test gene expression profile,
wherein a decrease in the CCR1, CCRL1, CMKLR1, IL8Rα, RGS7, 5-HT_{2A}, 5-HT₇ and PKCβ2 gene expression levels at said second or third instant relative to said first instant; together with an increase in the CCR5 and GABA_{A}β2 gene expression levels at said second or third instant relative to said first instant; and together with an increase in CCR7 and CCRL1 gene expression levels at said third instant relative to said second instant, indicate that said drug or drug combination has a likelihood of being effective in treatment of said patient.

26. A kit for evaluating the pharmacological efficacy of a drug candidate in treatment of a psychiatric disease or disorder; or for predicting the efficacy of a drug or drug combination indicated for treatment of both positive and negative symptoms of psychiatric diseases or disorders in a patient having a psychiatric disease or disorder, said kit comprising:
(i) a list of genes expressed in peripheral mononuclear cells (PMCs);
(ii) a predetermined reference gene expression profile obtained from a group of patients administered with a drug or drug combination effective against both positive and negative symptoms of psychiatric diseases or disorders by measuring expression levels of said genes in blood samples obtained from said patients at a first instant before the first administration of said drug or drug combination and at given second and third instants following the first administration of said drug or drug combination, said profile expressing a representative relative level of each one of said genes at said second and third instants for said group of patients, indicating an effective treatment against both positive and negative symptoms of psychiatric diseases or disorders;
(iii) a set of oligonucleotides each comprising a nucleotide sequence complementary to a specific sequence of each one of said genes;
(iv) instructions for use; and optionally
(v) a container containing said drug or drug combination.
